(19) United States
(12) Patent Application Publication
(10) Pub. No.: US 2016/0159778 A1
(43) Pub. Date: Jun. 9, 2016

(54) NOVEL UREIDO DERIVATIVES OF NAPHTHALENESULFONIC ACIDS

(71) Applicant: NeED PHARMACEUTICALS S.r.l., Milano MI (IT)

(72) Inventors: Nicola MONGELLI, Milano MI (IT); Paolo COZZI, Milano MI (IT); Prishilla BISWAS, Milano MI (IT); Daniela JABES, Milano MI (IT)

(21) Appl. No.: 14/906,096

(22) PCT Filed: Jul. 16, 2014
(86) PCT No.: PCT/IB2014/001335

§ 371 Date: Jan. 19, 2016

(30) Foreign Application Priority Data
Jul. 19, 2013 (IT) MI2013A001218

(51) Int. Cl. C07D 403/14; C07D 209/42

(52) U.S. Cl. CPC C07D 403/14; C07D 209/42

(57) ABSTRACT

Objects of the present invention are novel ureido derivatives of naphthalensulfonic acids, pharmaceutical compositions comprising such derivatives, a process for the preparation thereof and the use of said derivatives as medications, in particular in the treatment of HIV infections. Formula (I):

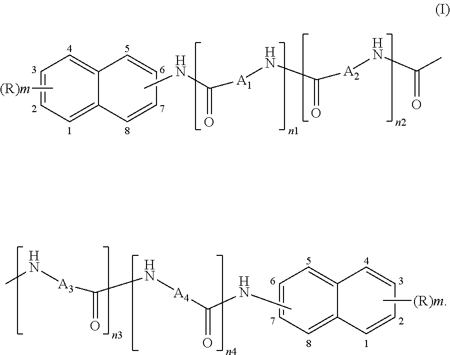

### FIELD OF THE INVENTION

Since 1981, year of the discovery of AIDS (Acquired Immuno Deficiency Syndrome), there are at present an estimated 33.4 million people worldwide that are living with HIV (Human Immunodeficiency Virus)/AIDS, with 2.7 million new HIV infections per year and 2.0 million deaths yearly due to AIDS (AIDS epidemic update, WHO 2009). According to the UNAIDS report in 2009, there have been about 60 million people infected worldwide since the beginning of the pandemic, with about 25 million deaths and 14 million orphaned children in the South African territory only (UNAIDS. 09 GLOBAL FACTS & FIGURES—Unaids).

The first drug able to stem the effects of the syndrome, was zidovudine (or AZT), a reverse transcriptase inhibitor, available since 1987. The tendency of the virus to develop resistant mutations and the high toxicity of the drug led to abandon the monotherapy in 1991, following the commercialization of a novel therapy with two drugs (bitherapy). Finally, since 1996, the discovery of protease inhibitors (N Engl J Med 338 (13): 853-60) allowed a new highly effective pharmaceutical protocol, based on three viral inhibitors (tritherapy), representing the current standard therapy, called HAART (Highly Active Antiretroviral Therapy).

Use of HAART has significantly impacted the AIDS epidemic, primarily in industrialized countries. However, the limit of antiretroviral therapy is exacerbated by the complications of the treatment regimens, by the development of resistant variants among isolated strains of HIV-1 (Clavel F and Hance A J: N. Engl. J. Med. 2004, 350, 1023-1035), and a number of side effects (Deeks S G et Al, JAMA 1997, 277, 145-153).

There are four attack strategies of the viral cycle that are taken into account in studies designed to identify new drugs:
i) virus attack and entry into the host cell, that occurs by a surface protein known as CD4, present on the surface of some immune cells; ii) reverse transcription and integration, using viral RNA to synthesize single stranded DNA, that is then duplicated, migrates in the core of the host cell and integrates in its genome;
iii) transcription and translation, when the pro-viral DNA is transcripted in messenger DNA and subsequently translated in viral proteins, which will then be modified by a HIV protease;
iv) assembling and release, when the viral proteins are assembled in the cytoplasm and new viral particles are released by the cellular surface to the circulation.

Many drugs, currently in the market, act both on the reverse transcriptase and protease. However, these treatments aren't well tolerated in addition to that concerns on the long term metabolic effects of the protease inhibitors, in particular on fat metabolism, increase more and more.

Today, the typical therapy consists in administering two nucleoside reverse transcriptase inhibitors (NRTI) plus one protease inhibitor or one non-nucleoside reverse transcriptase inhibitor (NNRTI). Since the HIV disease progression in children is more rapid than in adults, for the first protocols a more aggressive treatment is provided (Working Group on Antiretroviral Therapy and Medical Management of HIV-Infected Children, Nov. 3, 2005). In developed countries, where HAART therapy is available, doctors assess the viral load, CD4 count, rate of decay of the latter and the clinical conditions of the patient prior to deciding when to start the treatment. Traditionally, the treatment has been advised to asymptomatic patients when the CD4 cell count was lower than 200-250 units per milliliter of blood. However, starting the treatment earlier (at CD4 level of 350 cells/μl) can significantly reduce the risk of death.

The standard targets of HAART include the improvement of the quality of patient's life, reduction of complications and viremia reduction below the detection limit. Nevertheless, the therapy does not imply neither the treatment of the disease nor prevents the return, once the treatment is interrupted, of high blood levels of HIV, which is often resistant to further therapy cycles (Proc Natl Acad Sci USA 97 (20): 10948-53; Ann. Intern. Med. 137 (5 Pt 2): 381-433).

Notwithstanding this, many individuals infected with HIV have benefited from significant improvements in their general state of health and quality of life, with a strong decrease in the morbidity and mortality associated with the HIV virus (N. Engl. J. Med. 338 (13): 853-860; AIDS 17 (5): 711-720). In the absence of HAART therapy, the progression from HIV infection to AIDS occurs in a median comprised between 9 and 10 years whereas the median survival, after developing AIDS, is 9.2 months only (AIDS 16 (4): 597-632). With these premises, the identification of new classes of drugs active on HIV that are characterized by new action mechanisms and a favorable toxicological profile, represents an essential goal.

After the identification of CD4 as the main receptor for the HIV virus entry into the immune cells, soon it became apparent that CD4 alone could not establish a productive infection. The trans-membrane receptors of CXCR4 and CCR5 chemokines have subsequently been identified as co-receptors for the HIV-1 entry (Bleul C C et Al. Nature 1996, 382, 829-833). HIV-1 infection starts from the attack of the viral capsid glycoprotein, gp120, at CD4 on the host cell. The binding to CD4 activates a conformational change in gp120, that exposes a binding site for a chemokine receptor acting as co-receptor (Trkola A et Al, Nature 1996, 384, 184-187). The interaction with the co-receptor activates a rearrangement of the trans-membrane subunit of the capsid glycoprotein, gp41, that leads to fusion among virus and cell membranes. The most important chemokine receptors used as co-receptors for isolated HIV-1 entry, are CCR5 and CXCR4. CCR5 is today considered the most important co-receptor for the strains with tropism for macrophages, which are normally transmitted among individuals, whereas CXCR4 is considered the most important co-receptor for isolates with tropism for T lymphocytes, emerging many years after infection. Furthermore, the block of CCR5 function does not necessarily mean health effects on the individual, since about 1% of Caucasians lack of this co-receptor due to a mutation destroying the protein, without apparent consequences. Thus, the two HIV-1 co-receptors CCR5 and CXCR4 represent novel therapeutic targets for developing novel anti-HIV therapies.

Pharmaceutical industries investigated many CCR5 antagonists, but only a few have been tested on humans. For example, AstraZeneca, Novartis, Merck and Takeda evaluated wide molecule collections for developing potent CCR5 inhibitors, but none of these inhibitors has never been tested on humans (Bioorganic & Medicinal Chemistry Letters 15 (22): 5012-5, Bioorganic & Medicinal Chemistry Letters 11 (2): 259-64; Journal of Medicinal Chemistry 47 (8): 1939-55; Antimicrobial Agents and Chemotherapy 49 (8): 3483-5).

Among the three products that have been evaluated in clinical studies on humans, Aplaviroc (GSK), Vicriviroc (Schering-Plough) and Maraviroc (Pfizer), only the latter has been approved by FDA.

In about 50% of cases of infected individuals, viruses able to use CXCR4 in addition to CCR5 in association with the disease progression, have been identified. However viruses that use CXCR4 only are rather rare (about 1%). Thus, in these cases, a drug acting on CXCR4 may represent an important added value for those patients infected with CXCR4 tropic ism virus, both from the beginning of the therapy and in combination with HAART, independently of the presence of a CCR5 inhibitor.

Thus, an active compound inhibiting both co-receptors may represent a huge therapeutic advantage, since it would bring viruses using both co-receptors under control, by limiting the appearance of viruses preferentially using CXCR4 in those subjects infected by CCR5 tropic virus.

As previously mentioned, the standard therapy for the treatment of HIV infection currently provides for simultaneous administration of a number of drugs, mainly orally, with the necessity, by the patient, to ingest a high number of tablets many times a day. This regimen can be an important limit for the patient who, to properly adhere to the protocol, have to follow complex daily schemes that, if not respected, can bring to therapeutic inefficacy or to a selection of resistant viral strains. It must not be forgotten that patients with AIDS often suffer from spread mucosites, in particular in the oral cavity, due to fungal infections, which may impede or however hinder and make swallowing tablets problematic. Alternative routes of administration of novel anti-HIV drugs thus represent an important contribution to increase the patient adhesion to the therapeutic protocol, with evident positive consequences on the effectiveness of the therapy itself.

Thus, purpose of the present invention is to provide an active compound which could be employed in anti-HIV therapy and have easiness of administration.

Further purpose of the present invention is to provide an active compound which could be employed in anti-HIV therapy that is highly effective in inhibiting co-receptor CXCR4 in addition to inhibit CCR5.

Still purpose of the invention is to provide an active compound which could be employed in anti-HIV therapy which is characterized by a good toxicological profile and thus being much more tolerable than the drugs currently available in the market.

These and still other purposes and relative advantages which will be better explained by the following description are achieved by the compounds according to the invention, having the following general formula (I):

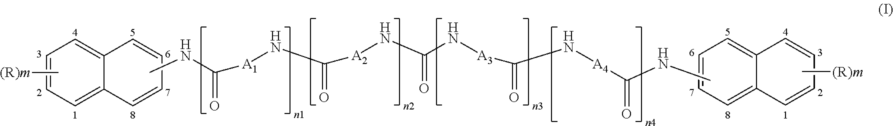

where:
   A1, A2, A3, A4 are independently selected from: pyrrole, pyrazole or indole, provided that at least one of said A1, A2, A3 and A4 is an indole;
   n1, n2, n3, n4 are independently selected to be equal to 0, 1, 2 provided that the sum of said n1, n2, n3, n4 is comprised between 2 and 6;
   R is a sulfonic acid residue;
   m is an integer comprised between 1 and 3.

Also pharmaceutically acceptable salts of said compounds of formula (I) are object of the invention. Examples of pharmaceutically acceptable salts are, for example, those from compounds of formula (I) and inorganic bases, such as sodium, potassium, calcium and aluminum hydroxides, as well as those with organic bases such as, for example, lysine, arginine, N-methyl-glucamine, triethylamine, triethanolamine, dibenzilamine, methylbenzilamine, d-(2-ethyl-hexyl)-amine, piperidine, N-ethylpiperidine, N,N-di-ethylaminoethylamine, N-ethylmorpholine, 3-phenethylamine, N-benzil-3-phenethylamine, N-benzil-N,N-dimethylamine.

Compounds of formula (I) are urea derivatives of the amino-naphthalensulfonic acid and are advantageously employed as active compounds with antiviral activity, in particular against the acquired human immunodeficiency virus (HIV).

Preferred compounds, according to the present invention, are those belonging to the general formula (I) where:

n1, n2, n3, n4 are independently selected from 0 and 1, provided that the sum of said n1, n2, n3, n4 is comprised between 2 and 4;
A1, A2, A3, A4 are selected from pyrrole and indole, provided that at least one of said A1, a2, A3, A4 is an indole;
R is a sulfonic acid residue;
m is an integer between 1 and 3.

Specific examples of the preferred compounds according to the invention, are the following:
7-{[(4-{[5-({[2-({5-[(6,8-disulfonaphthalen-2-yl)carbamoyl]-1-methyl-1H-pyrrol-3-yl}carbonyl)-1-methyl-1H-indol-5-yl]carbamoyl}amino)-1-methyl-1H-indol-2-yl]carbonyl}-1-methyl-1H-pyrrol-2-yl)carbonyl]amino}naphthalene-1,3-disulfonic acid
7-[({5-[({2-[(6,8-disulfonaphthalen-2-yl)carbamoyl]-1-methyl-1H-indol-5-yl}carbamoyl)amino]-1-methyl-1H-indol-2-yl}carbonyl)amino]naphthalene-1,3-disulfonic acid
7-({[5-({[4-({[5-({2-[(6,8-disulfonaphthalen-2-yl)carbamoyl]-1-methyl-1H-indol-5-yl}carbamoyl)-1-methyl-1H-pyrrol-3-yl]carbamoyl}amino)-1-methyl-1H-pyrrol-2-yl]carbonyl}amino)-1-methyl-1H-indol-2-yl]carbonyl}amino)naphthalene-1,3-disulfonic acid
4-({[4-({[5-({[2-({5-[(4,6-disulfonaphthalen-1-yl)carbamoyl]-1-methyl-1H-pyrrol-3-yl}amino)-1-methyl-1H-indol-5-yl]carbamoyl}amino)-1-methyl-1H-indol-2-yl]carbonyl}amino)-1-methyl-1H-pyrrol-2-yl]carbonyl}amino)naphthalene-1,7-disulfonic acid
7-({[1-methyl-4-({[1-methyl-5-({[1-methyl-2-({1-methyl-5-[(4,6,8-trisulfonaphthalen-2-yl)carbamoyl]-1H-pyrrol-3-yl}carbamoyl)-1H-indol-5-yl]carbamoyl}amino)-1H-indol-2-yl]carbonyl}amino)-1H-pyrrol-2-yl]carbonyl}amino)naphthalene-1,3,5-trisulfonic acid
   and pharmaceutically acceptable salts thereof, in particular sodium and potassium salts.

Particularly preferred, according to the present invention, is the compound having the following formula (VII):

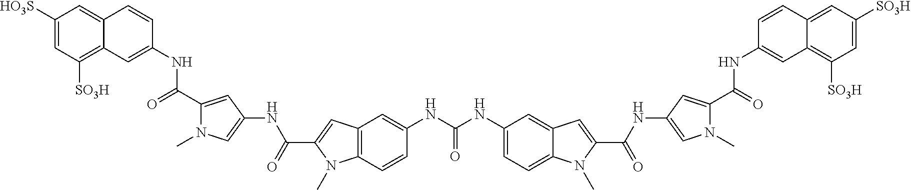 [SMILES: Cn1cc(NC(=O)c2cc3cc(NC(=O)Nc4ccc5c(c4)cc(C(=O)Nc4cc(C(=O)Nc6ccc7cc(S(=O)(=O)O)cc(S(=O)(=O)O)c7c6)n(C)c4)n5C)ccc3n2C)cc1C(=O)Nc1ccc2cc(S(=O)(=O)O)cc(S(=O)(=O)O)c2c1]

### 7-({[4-({[5-({[2-({5-[(6,8-Disulfonaphthalen-2-yl)carbamoyl]-1-methyl-1H-pyrrol-3-yl amino)-1-methyl-1H-indol-5-yl]carbamoyl}amino)-1-methyl-1H-indol-2-yl]carbonyl}amino)-1-methyl-1-H-pyrrol-2-yl]carbonyl}amino)naphthalen-1,3-disulfonic acid

Compounds according to the invention and corresponding salts thereof, can be prepared by a process including reacting a compound of formula (II) as herein indicated:

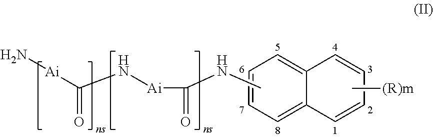

where Ai can independently be A1, A2, A3 or A4 as defined above and ns can independently be n1, n2, n3 or n4 as defined above, or a corresponding salt thereof, with a compound of formula (III) as herein reported:

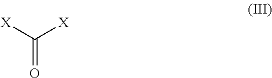

where each of the X groups, which can be the same or different from each other, is selected with the characteristics of a good leaving group. At the end of the reaction between the compound of formula (II) and that of formula (III), if required, it is possible to proceed to the salification of the so-obtained compound of formula (I). In addition, it is possible to obtain the compound of formula (I) in a free form by the corresponding salt.

Also in case of compound of formula (II), the corresponding salts can be salts with inorganic bases, for example those mentioned above, whereas for what concerns pharmaceutically acceptable salts, sodium and potassium salts are preferred.

For what concerns the terminology relative to “good leaving group” as indicated above on the occasion of the definition of substituent X in formula (III), such a terminology means groups selected, for example, from halogen atoms such as in particular chlorine, or else other groups easily substitutable such as, for example, imidazole, triazole, p-nitrophenyloxy, trichlorophenyloxy or trichloromethyloxy.

The reaction of the compound of formula (II) or a salt thereof with the compound of formula (III), can be accomplished according to known methods, in particular following the directions found in organic chemistry texts for the synthesis of urea derivatives. Preferably, when in compounds of formula (III) X is selected equal to halogen, for example chlorine, the reaction can be accomplished in a molar ratio between compound (II) or a salt thereof and compound (III) from about 1:1 to about 1:4.

The reaction is preferably accomplished in organic solvents such as dimethylacetamide, dimethylsulfoxyde, hexamethylphosphoramide, preferably dimethylformamide, or aqueous mixtures thereof, or else water/dioxane or water/toluene mixtures thereof, both in presence of an organic base such as for example triethylamine or diisopropylethylamine and in presence of an inorganic base, such as for example sodium bicarbonate or sodium acetate.

The reaction temperature can vary between about −10° C. and about 50° C. and the reaction times can vary between about 1 hour and about 12 hours.

The compounds of formula (I), prepared according to the processes indicated above, can be purified following conventional methods such as silica gel or alumina column chromatography and/or by recrystallization from organic solvents such as, for example, lower-chain aliphatic alcohols or dimethylformamide.

Similarly, the salification of compounds of formula (I) can be accomplished according to known techniques.

Also the compounds of formula (II) can be prepared following the known art.

For example, a compound of formula (II) can be synthesized, according to what is known to the ordinary technician in the field, by a reduction reaction of a compound having the formula (IV) reported hereinbelow:

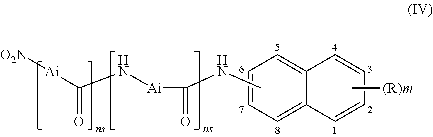

where ns and Ai are as defined above.

In turn, the compounds of formula (IV) can be obtained by reacting an amine of formula (V)

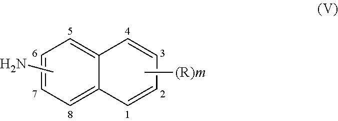

where (R)m is defined as above, with a compound of formula (VI)

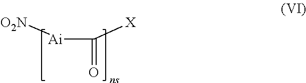

where Ai, ns and X are defined as above.

The reaction between the amine of formula (V) and the compound of formula (VI) is also well known in the state of the art.

The invention will be better described by the following examples of practical implementation, which are provided for illustration purposes only and non-limiting the present invention.

### EXAMPLE 1

### Preparation of the tetrasodium salt of the 7-{[(4-{[5-({[2-({5-[(6,8-di sulfonaphthalen-2-yl)carbamoyl]-1-methyl-1H-pyrrol-3-yl}carbonyl)-1-methyl-1H-indol-5-yl]carbamoyl}amino)-1-methyl-1H-indol-2-yl]carbonyl}-1-methyl-1H-pyrrol-2-yl)carbonyl]amino naphthalene-1,3-disulfonic acid

To a solution of 600 mg (1 mmole) of 7-{[(1-methyl-4-{[(1-methyl-5-amino-1H-indol-2-yl)carbonyl]amino}-1H-pyrrol-2-yl)carbonyl]amino}naphthalene-1,3-disulfonic acid in a water/dioxane mixture (20 ml, 1/1 v/v) containing 350 mg (4 mmoles) of NaHCO₃, 55 mg (0.2 mmoles) bis(trichloromethyl)carbonate was added dropwise and under stirring. The resulting mixture was left under stirring for 8 hours at room temperature. 20 ml of dioxane was added, and the so-obtained precipitate was filtered and washed, giving 850 mg, (yield 70%) of a red powder. ¹H NMR (DMSO-d₆) d ppm 3.62 (s, 3H) 3.79 (s, 3H) 3.82 (s, 3H) 3.86 (s, 3H) 6.25 (s, 1H) 6.57 (s, 1H) 6.57 (s, 1H) 6.88 (s, 1H) 7.00 (d, J=8.68 Hz, 1H) 7.01 (s, 1H) 7.22 (s, 1H) 7.29 (d, J=8.68 Hz, 1H) 7.38 (d, J=8.68 Hz, 1H) 7.50 (d, J=8.68 Hz, 1H) 7.54 (s, 1H) 7.82 (s, 1H) 8.21 (s, 1H) 8.21 (s, 1H) 8.44 (sc, J=8.58 Hz, 1H) 8.44 (sc, J=8.58 Hz, 1H) 8.46 (sc, J=8.58 Hz, 1H) 8.46 (sc, J=8.58 Hz, 1H) 8.73 (s, 1H) 8.73 (s, 1H) 8.87 (s, 1H) 8.87 (s, 1H)

F.A.B. -M.S. Mlz 1194; M′+1; 1216, M++23;

Following a similar procedure, the following compounds are obtained:
tetrasodium salt of the 7-({[5-({[4-({[5-({2-[(6,8-disulfonaphthalen-2-yl)carbamoyl]-1-methyl-1H-indol-5-yl}carbamoyl)-1-methyl-1H-pyrrol-3-ylicarbamoyl}amino)-1-methyl-1H-pyrrol-2-yl]carbonyl}amino)-1-methyl-1H-indol-2-yl]carbonyl}amino)naphthalene-1,3-disulfonic acid
tetrasodium salt of the 4-([4-({[54 {[2-({5-[(4,6-disulfonaphthalen-1-yl)carbamoyl]-1-methyl-1H-pyrrol-3-yl}amino)-1-methyl-1H-indol-5-yl]carbamoyl}amino)-1-methyl-1H-indol-2-yl]carbonyl}amino)-1-methyl-1H-pyrrol-2-yl]carbonyl}amino)naphthalene-1,7-disulfonic acid
hexasodium salt of the 7-({[1-methyl-4-({[1-methyl-54 {[1-methyl-2-({1-methyl-5-[(4,6,8-trisulfonaphthalen-2-yl)carbamoyl]-1H-pyrrol-3-yl}carbamoyl)-1H-indol-5-yl]carbamoyl}amino)-1H-indol-2-yl]carbonyl}amino)-1H-pyrrol-2-yl]carbonyl}amino)naphthalene-1,3,5-trisulfonic acid

### EXAMPLE 2

### Preparation of the tetrasodium salt of the 7-[({5-[({2-[(6,8-disulfonaphthalen-2-yl)carbamoyl]-1-methyl-1H-indol-5-yl}carbamoyl)amino]-1-methyl-1H-indol-2-yl}carbonyl)amino]naphthalene-1,3-disulfonic acid

To a solution of 475 mg (1 mmole) of tetrasodium salt of the 7-{[(5-amino-1-methyl-1H-indol-2-yl)carbonyl]amino}naphthalene-1,3-disulfonic acid in water (15 ml) and dioxane (5 ml), NaHCO₃ (350 mg, 4 mmoles) was added, under stirring. The reaction mixture was cooled with an ice bath and then 55 mg bis(trichloromethylcarbonate) was added dropwise and under stirring, and the resulting mixture was maintained under stirring for 8 hours at room temperature. Afterwards, 25 ml dioxane was added, and a precipitate was obtained which was washed and filtered, giving 399 mg, (yield 75%) of the desired compound as an orange powder.

1H NMR (400 MHz, Solvent) δppm 3.86 (s, 3H) 3.86 (s, 3H) 6.78 (s, 1H) 6.78 (s, 1H) 7.37 (sc, J=8.68 Hz, 1H) 7.38 (d, j=8.68 Hz, 1H) 7.38 (sc, J=8.68 Hz, 1H) 7.50 (d, J=8.68 Hz, 1H) 7.82 (s, 1H) 7.82 (s, 1H) 8.21 (s, 2H) 8.44 (sc, J=8.58 Hz, 1H) 8.44 (sc, J=8.58 Hz, 1H) 8.46 (sc, J=8.58 Hz, 1H) 8.46 (sc, J=8.58 Hz, 1H) 8.73 (s, 2H) 8.82 (s, 2H)

F.A.B. -M.S. Mlz 977; M′+1; 999, M++23;

Following a similar procedure, also the following compounds were obtained:
tetrasodium salt of the 4-[({5-[({2-[(4,6-disulfonaphthalen-1-yl)carbamoyl]-1-methyl-1H-indol-5-yl}carbamoyl)amino]-1-methyl-1H-indol-2-yl}carbonyl)amino]naphthalene-1,7-disulfonic acid
hexasodium salt of the 7-[({1-methyl-5-[({1-methyl-2-[(4,6,8-trisulfonaphthalen-2-yl)carbamoyl]-1H-indol-5-yl}carbamoyl)amino]-1H-indol-2-yl}carbonyl)amino]naphthalene-1,3,5-trisulfonic acid

### EXAMPLE 3

### Preparation of the 7-[({4-[(5-amino-1-methyl-1H-indol-2-yl)carbonyl]-1-methyl-1H-pyrrol-2-yl}carbonyl)amino]naphthalene-1,3-disulfonic acid

To a solution of 630 mg (1 mmole) of 7-{[(1-methyl-4-{[(1-methyl-5-nitro-1H-indol-2-yl)carbonyl]amino}-1H-pyrrol-2-yl)carbonyl]amino}naphthalene-1,3-disulfonic acid in 40 ml water, hydrochloric acid 1M (1 mmole) and 10% Pd on carbon were added. The suspension was subjected to hydrogenation reaction at 50 psi at room temperature for 8 h and then was filtered and washed thereby giving, after solvent evaporation, 7-{[(1-methyl-4-[(1-methyl-5-amino-1H-indol-2-yl)carbonyl]amino}-1H-pyrrol-2-yl)carbonyl]amino}naphthalene-1,3-disulfonic acid (580 mg, yield 97%).

1H NMR (400 MHz, Solvent (DMSO-d6) δ ppm 3.70-3.97 (m, 8H) 6.36-6.63 (m, 3H) 6.84 (s, 1H) 7.01 (s, 1H) 7.14 (d, J=8.69 Hz, 2H) 7.22 (s, 1H) 8.21 (s, 1H) 8.25-8.80 (m, 4H) 8.87 (s, 2H).

Following a similar process, the following compounds were obtained:
7-{[(5-amino-1-methyl-1H-indol-2-yl)carbonyl]amino}naphthalene-1,3-disulfonic acid
7-{[(4-amino-1-methyl-1H-pyrrol-2-yl)carbonyl]amino}naphthalene-1,3-disulfonic acid

### EXAMPLE 4

### Preparation of the 7-{[(1-methyl-4-{[(1-methyl-5-nitro-1H-indol-2-yl)carbonyl]amino}-1H-pyrrol-2-yl)carbonyl]amino}naphthalene-1,3-disulfonic acid

To a cooled solution of 450 mg (1 mmole) of 7-{[(4-amino-1-methyl-1H-pyrrol-2-yl)carbonyl]amino}naphthalene-1,3-disulfonic acid in a water-dioxane mixture (10 ml, 1/4 v/v) containing NaHCO₃ (2 mmoles), 1-methyl-5-nitro-1H-indole-2-carbonyl chloride (1 mmole) in dioxane (3 ml) was added dropwise. After 2 h at 0° C., the reaction temperature was allowed to raise to room temperature and left under stirring for 6 hours. The resulting precipitate was filtered, washed with dioxane and dried, giving a red powder (510 mg, yield 80%).

1H NMR (400 MHz, Solvent) δ ppm 3.82 (s, 3H) 3.88 (s, 3H) 7.01 (s, 1H) 7.05 (s, 1H) 7.22 (s, 1H) 7.54 (d, J=9.00 Hz, 1H) 8.04 (d, J=9.00 Hz, 1H) 8.18 (d, J=8.63 Hz, 1H) 8.37 (d, J=8.63 Hz, 1H) 8.37 (s, 1H) 8.47 (s, 1H) 8.63 (s, 1H) 9.17 (s, 1H).

### EXAMPLE 5

### Preparation of the 7-{[(1-methyl-5-nitro-1H-indol-2-yl)carbonyl]amino}naphthalene-1,3-disulfonic acid

To a solution maintained under stirring of disodium salt of the 7-aminonaphthalene-1,3-disulfonic acid (192 mg, 0.5 mmoles) in 20 ml water/dioxane 1/1 v/v and 0.46 ml 1N NaOH, 180 mg 1-methyl-5-nitro-1H-indol-2-carbonyl chloride in 2 ml dioxane was added dropwise. The desired compound was precipitated by addition of 10 ml dioxane after 2 hours at room temperature.

### EXAMPLE 6

### Preparation of 1-methyl-5-nitro-1H-indole-2-carbonyl chloride

A suspension of 220 mg (1 mmole) of 1-methyl-5-nitro-1H-indol-2-carboxylic acid (currently commercially available as Capot™) in 3 ml anhydrous dioxane containing 0.5 ml SOCl₂, was maintained boiling under reflux. The solvent was then evaporated and the residue was used without further purifications.

The following compounds are obtained by similar processes:

### 1-methyl-4-nitro-1H-pyrrol-2-carbonyl chloride

The compounds of general formula (I) object of the present invention are thus novel HIV virus inhibitors and act by exerting their inhibitory action against the virus entry into the cell, by binding to both CXCR4 and CCR5 co-receptors. Furthermore, compounds object of the present invention are characterized by a very peculiar pharmacokinetics since, if intravenously administered, they induce an extremely prolonged plasma exposure. This aspect is particularly advantageous because it involves the possibility to carry out a unique administration to be repeated in 2-3 weeks' time. Such a characteristic is an obvious advantage, not only in terms of adhesion to the treatment by the patient who therefore is no longer required to orally take other drugs, in addition to the already numerous necessarily administered, but also implies a consequent greater probability of effectiveness and a consequent reduction of the risk to select resistant strains.

Thus, the compounds subject of the present invention can be administered in a single dosage to be repeated not more than twice a month. This figure has to be compared with the dosage of the pharmacological therapies currently in use, that provide for oral and daily drug administration. Since these are multi-drug therapies, the patient, as already mentioned, has to take several caplets every day, with obvious problems and complications which are also logistical and practical. By using the active compound according to the present invention, conversely it is possible, at least for what concerns the therapeutic category to which the mentioned active compound is subjected, to avoid both oral and daily administration, thereby limiting the intake once every two weeks and using the administration alternative to the oral one.

With reference to what described above, the following examples are reported still by way of example only and non-limiting the present invention.

### EXAMPLE 7

The compound (VII) has been tested for its ability to modulate the viral replication of HIV-1, both of a strain using CXCR4 (IIIB) and a strain using CCR5 (Bal) as co-receptor, in addition to the primary receptor CD4.

For the in-vitro infections primary cells have been used, specifically peripheral blood mononuclear cells from healthy donors pre-activated for 48 hours through a mitogenic stimulus (phytohemagglutinin) to allow for the infection by HIV-1. The cells have been incubated with IIIB and Bal viruses in absence or in presence of scalar concentrations of compound (VII) for 30 minutes at 37° C., then they have been washed to remove the excess virus, plated in triplicate in culture microplates and the compound VII has been re-added to the wells, in the test concentrations. The control culture has been set up in presence of dimethylsulfoxyde (DMSO), the compound in which the compound VII has been dissolved, in order to more correctly evaluate a net effect of the compound. The viral replication has been evaluated by measuring the structural protein of HIV-1 p24 (expressed in ng/ml) by an immune-enzymatic test carried out on the culture supernatants harvested on days 4 and 7 post-infection.

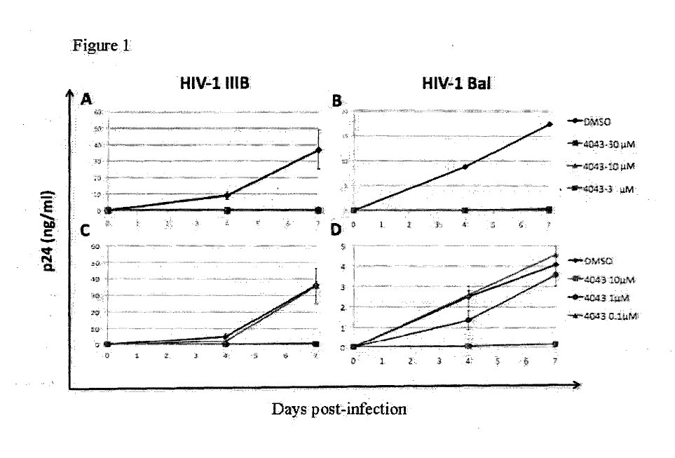

FIG. 1 shows the results of two independent experiments: in the first one, the compound (VII) according to the invention has been tested at 30, 10 and 3 μM (FIG. 1 A/B), in the second one at 10, 1 and 0.1 μM (FIG. 1 C/D). The graphs show the mean values of the culture triplicate with the standard deviation on post-infection days 4 and 7.

The compound (VII) of the invention completely inhibited the replication both of HIV-1 IIIB and Bal at concentrations of 30, 10 and 3 μM (FIG. 1A/B). The concentration 0.1 μM was not more effective, whereas the compound (VII) at 1 μM still inhibited completely IIIB, whereas it almost totally lost the inhibitory ability against Bal (FIG. 1 C/D). This figure underlines the higher effectiveness of the compound against viruses using the chemokine receptor CXCR4 as a co-receptor.

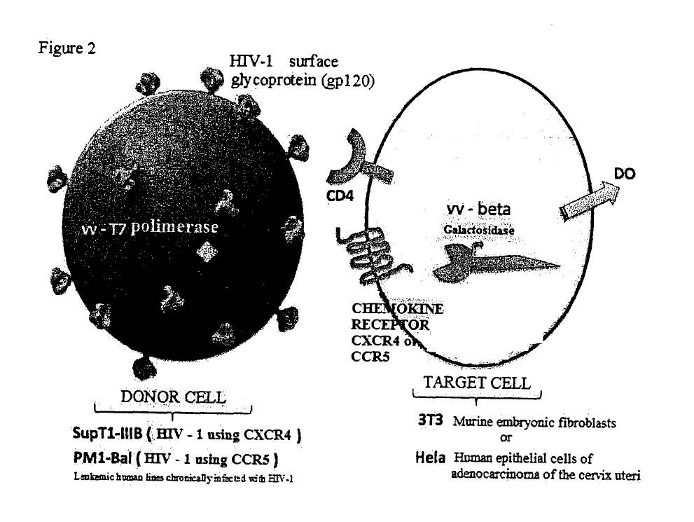

In order to evaluate on which step of the complex HIV-1 replication cycle the compound VII acts according to the present invention, a sophisticated method, schematized in FIG. 2, has been used, specifically only focusing on the entry step of HIV-1 in target cells.

Briefly, a colorimetric reaction is developed when two populations of properly engineered cells meet and in particular a donor, carrying on the surface the glycoprotein of HIV-1 gp120 and a target, together with the primary receptor CD4 and with one of the co-receptors, CXCR4 or CCR5. In this case there is a fusion of the cells and the polymerase T7 can pass from the donor cell to the target one in which it triggers the transcription of beta-galactosidase with subsequent protein translation. Thus the latter is able to perform its enzymatic action in the presence of the convenient substrate (CPRG, chlorophenol red-3-D galactopyranoside) developing a colorimetric reaction measured by detection of optical density (OD) at 570 nm.

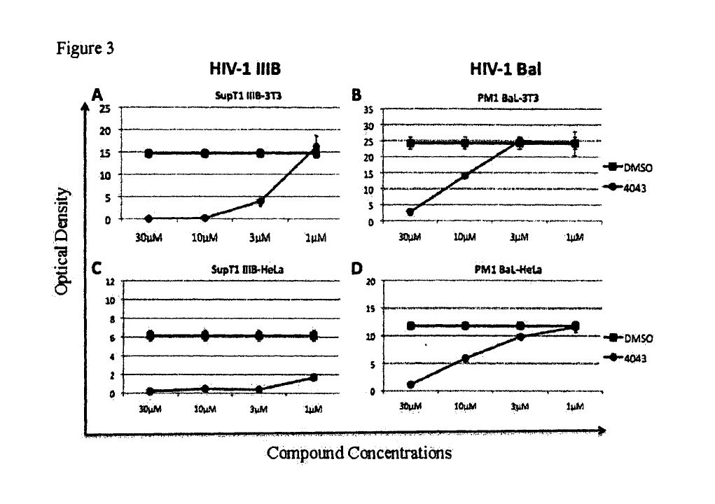

The results of two independent experiments are shown in FIG. 3. In the first one, the target cells are mouse fibroblasts (3T3 in FIG. 3 A/B), on the contrary in the second one the target cells are human epithelial cells (HeLa, in FIG. 3 C/D). The graphs show the mean values of the culture triplicate with the standard deviation. It can be noticed that the compound (VII) according to the present invention is able to inhibit, in a way depending on the concentration, the fusion mediated by gp120 both of HIV-1 using CCR5 (FIG. 3 B/D) and HIV-1 using CXCR4 (FIG. 3 A/C), however being more potent against the latter, in line with what observed in the above commented inhibition experiments of the viral replication.

Therefore, it can be stated that the compound (VII) according to the invention is characterized by a considerable inhibitory capacity against the HIV-1 replication and that this effect is achieved at the virus entry level into the target cells. Finally, the inhibitory effect is more accentuated against HIV-1 using the co-receptor CXCR4. Similar results, for what concerns the inhibitory capacity against the HIV-1 replication, have been observed for others among the synthesized compounds belonging to the general formula (I).

Thus, according to the present invention, the compounds of formula (I) and pharmaceutically acceptable salts thereof, are employed as medications, in particular they are employed as active compounds for the preparation of pharmaceutical compositions further comprising usual excipients of pharmaceutically acceptable type.

Thus, the compounds of general formula (I) and pharmaceutical compositions comprising them as active compounds, are employed in the treatment of HIV infections.

Furthermore, still according to the present invention, the compounds of general formula (I) and pharmaceutical compositions comprising them as active compounds, are also employed in bone marrow transplant, as well as in the treatment of tumoral states and in the treatment of inflammatory states.

Thus, the use of the compounds of general formula (I), and in particular of the preferred compound (VII), is aimed primarily at the treatment of HIV infections, in particular caused by HIV-1.

1. Compounds of general formula (I) 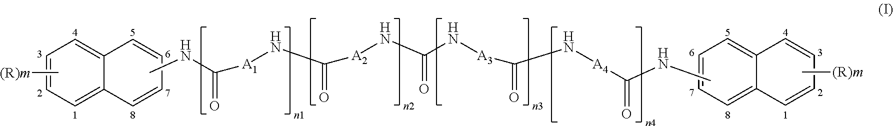
where:
A1, A2, A3, A4 are independently selected from: pyrrole, pyrazole or indole, provided that at least one of said A1, A2, A3 and A4 is an indole;
n1, n2, n3, n4 are independently selected to be equal to 0, 1, 2 provided that the sum of said n1, n2, n3, n4 is comprised between 2 and 6;
R is a sulfonic acid residue;
m is an integer comprised between 1 and 3.

2. Compounds according to claim 1, wherein:
n1, n2, n3, n4 are independently selected from 0 and 1, provided that the sum of said n1, n2, n3, n4 is comprised between 2 and 4;
A1, A2, A3, A4 are selected from pyrrole and indole, provided that at least one of said A1, a2, A3, A4 is an indole;
R is a sulfonic acid residue;
m is an integer between 1 and 3.

3. Compound according to claim 1, wherein it has the following formula (VII):

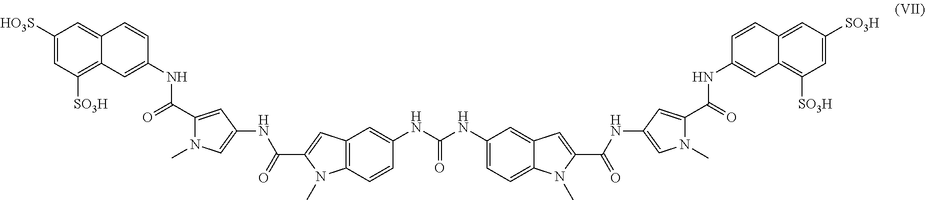 [SMILES: Cn1cc(NC(=O)c2cc3cc(NC(=O)Nc4ccc5c(c4)cc(C(=O)Nc4cc(C(=O)Nc6ccc7cc(S(=O)(=O)O)cc(S(=O)(=O)O)c7c6)n(C)c4)n5C)ccc3n2C)cc1C(=O)Nc1ccc2cc(S(=O)(=O)O)cc(S(=O)(=O)O)c2c1]

4. 7-({[4-({[5-({[2-({5-[(6,8-Disulfonaphthalen-2-yl)carbamoyl]-1-methyl-1H-pyrrol-3-yl}amino)-1-methyl-1H-indol-5-yl]carbamoyl}amino)-1-methyl-1H-indol-2-yl]carbonyl}amino)-1-methyl-1-H-pyrrol-2-yl]carbonyl}amino)naphthalen-1,3-disulfonic acid

5. Compound according to claim 1, wherein it is selected from:
7-{[(4-{[5-({[2-({5-[(6,8-disulfonaphthalen-2-yl)carbamoyl]-1-methyl-1H-pyrrol-3-yl}carbonyl)-1-methyl-1H-indol-5-yl]carbamoyl}amino)-1-methyl-1H-indol-2-yl]carbonyl}-1-methyl-1H-pyrrol-2-yl)carbonyl]amino}naphthalene-1,3-disulfonic acid;
7-[({5-[({2-[(6,8-disulfonaphthalen-2-yl)carbamoyl]-1-methyl-1H-indol-5-yl}carbamoyl)amino]-1-methyl-1H-indol-2-yl}carbonyl)amino]naphthalene-1,3-disulfonic acid;
7-({[5-({[4-({[5-({2-[(6,8-disulfonaphthalen-2-yl)carbamoyl]-1-methyl-1H-indol-5-yl}carbamoyl)-1-methyl-1H-pyrrol-3-yl]carbamoyl}amino)-1-methyl-1H-pyrrol-2-yl]carbonyl}amino)-1-methyl-1H-indol-2-yl]carbonyl}amino)naphthalene-1,3-disulfonic acid;
4-({[4-({[5-({[2-({5-[(4,6-disulfonaphthalen-1-yl)carbamoyl]-1-methyl-1H-pyrrol-3-yl}amino)-1-methyl-1H-indol-5-yl]carbamoyl}amino)-1-methyl-1H-indol-2-yl]carbonyl}amino)-1-methyl-1H-pyrrol-2-yl]carbonyl}amino)naphthalene-1,7-disulfonic acid;
7-({[1-methyl-4-({[1-methyl-5-({[1-methyl-2-({1-methyl-5-[(4,6,8-trisulfonaphthalen-2-yl)carbamoyl]-1H-pyrrol-3-yl}carbamoyl)-1H-indol-5-yl]carbamoyl}amino)-1H-indol-2-yl]carbonyl}amino)-1H-pyrrol-2-yl]carbonyl}amino)naphthalene-1,3,5-trisulfonic acid.

6. Compounds according to claim 1, wherein they are in the form of pharmaceutically acceptable salts.

7. Compounds according to claim 5, wherein said salts are sodium and potassium salts.

8. Process for preparing the compounds of claim 1, wherein it includes reacting a compound having the following formula (II): 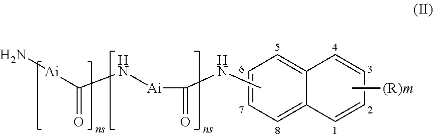
where Ai is independently selected as A1, A2, A3 or A4 and ns is independently selected as n1, n2, n3 or n4, or a corresponding salt thereof, with a compound having the following formula (III): 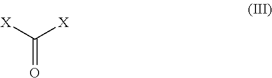
where each of said X, the same or different from each other, is selected from halogen, imidazole, triazole, p-nitrophenyloxy, trichlorophenyloxy, trichloromethyloxy.

9. Compounds of claim 1, for use as medications.

10. Compounds of claim 1, for use in the treatment of HIV infections.

11. Compounds of claim 1, for use in the treatment of tumor diseases.

12. Compounds of claim 1, for use in the treatment of inflammatory type diseases.

13. Compounds of claim 1, for use in the treatment of diseases related to bone marrow transplant.

14. Compound of claim 3, for use in the treatment of HIV infections.

15. Pharmaceutical compositions comprising at least one compound of claim 1 as an active compound, in addition to usual excipients of known type.

16. Pharmaceutical compositions comprising at least one compound of claim 3 as an active compound, in addition to usual excipients of known type.

17. Pharmaceutical compositions of claim 15 for use as a medication.

18. Pharmaceutical compositions of claim 15, for use in the treatment of HIV infections.

19. Pharmaceutical compositions of claim 15, for use in the treatment of HIV infections.
